(19) United States
(12) Patent Application Publication
(10) Pub. No.: US 2006/0182777 A1
(43) Pub. Date: Aug. 17, 2006

(54) Method of modulating drug release from a coated substrate

(76) Inventor: Steve Kangas, Woodbury, MN (US)

Correspondence Address: KENYON & KENYON LLP, 1500 K STREET N.W., SUITE 700, WASHINGTON, DC, 20005, US

(21) Appl. No.: 11/057,175

(22) Filed: Feb. 15, 2005

(51) Int. Cl. A61F 13/00; B05D 3/02

(52) U.S. Cl. 424/422; 427/2.24

(57) ABSTRACT

A method of modulating drug release from coated substrates by modulating the drying rate of the coatings on the substrates. Each coating is a mixture of a polymer, a solvent, and drug and the method includes modulating the release rate of the drug particles from the outer surface of the coatings by drying the solvent of each of the mixtures at different drying rates. A decrease in the drying rate of the solvent increases the initial release rate of the drug and an increase in the drying rate of the solvent decreases the initial release rate of the drug.

### FIELD OF THE INVENTION

The present invention is directed to a method of modulating the initial release of drug from the surface of coated substrates by modulating the drying rate of the coating of the substrates.

### BACKGROUND OF THE INVENTION

Minimally invasive medical devices such as stents, grafts, and balloon catheters, are used for a number of medical purposes. It is often beneficial to add coatings containing drugs to such medical devices to provide desired therapeutic properties and effects. For example, it is useful to apply a coating containing drugs to medical devices to provide for the localized delivery of drugs to target locations within the body. Compared to systemic drug administration, such localized drug delivery minimizes unwanted effects on parts of the body that are not to be treated and allows for the delivery of higher amounts of drugs to the afflicted part of the body.

An important consideration in the manufacture of medical devices having a coating containing drugs is obtaining the desired release rate of the drugs from the coating, particularly the desired release rate of the drugs at the surface of the coating. It is the drug particles that are at least partially exposed at the surface of the coating (as opposed to being embedded in the coating) that are initially released from the coating.

Current factors that affect drug release and that are therefore modulated during the medical device development process to modulate drug release from a coating include polymer characteristics, drug loading, solvent selection, and variables in the coating spray process such as solution flow rate, nitrogen pressure, temperature, and humidity. For coatings applied by a spray process, varying any of the spray process factors within current manufacturing limits typically has a relatively small impact on the kinetic drug release of the drug. Currently, the primary way to substantially modulate the kinetic drug release of drug particles from a coating is to modulate the amount of drug in the coating. However, simply adding more or less drug to the coating to affect the rate of initial drug release from the surface of the coating can create unwanted effects on the subsequent release of drug embedded in the polymer matrix of the coating, such as higher or lower drug release than desired. Furthermore, adding more drug to the coating may not be a cost-efficient mechanism to increase the initial drug release considering the high cost of many of the drugs that are incorporated into the coating. Accordingly, there is a need in the art for a more efficient and precise method of modulating the rate of initial drug release from the surface of coatings.

### SUMMARY OF THE INVENTION

The present invention provides a method of modulating drug release from coatings on substrates. The method comprises providing substrates and preparing mixtures, each of the mixtures comprising a polymer, a solvent, and drug. The method further comprises applying each of the mixtures to respective ones of the substrates to form coatings on the substrates, each of the coatings having an outer surface. The method further comprises modulating the release rate of drug from the outer surface of the coatings by drying the solvent of each of the mixtures at different drying rates.

### BRIEF DESCRIPTION OF THE DRAWINGS

The present invention will become more fully understood from the detailed description given hereinbelow and the accompanying drawings which are given by way of illustration only and wherein:

FIG. 1 is a schematic illustration of a substrate having a coating containing drug particles on the outer surface thereof.

FIG. 2 is an atomic force microscopy image of a substrate coated with a coating comprising 70/30 toluene/THF and wherein the coating has not been exposed to forced air.

FIG. 3 is an atomic force microscopy image of a substrate coated with a coating comprising 100% THF and wherein the coating has been exposed to forced air.

FIG. 4 is an atomic force microscopy image of a substrate coated with a coating comprising 50/50 toluene THF and wherein the coating has been exposed to forced air.

FIG. 5 depicts a chart of paclitaxel particle diameter on the surface of a substrate versus cumulative release of paclitaxel after a 24 hour period.

FIG. 6 depicts a chart of percent cumulative drug release over a three day period.

FIG. 7 depicts a plot of paclitaxel particle diameter versus particle count.

### DETAILED DESCRIPTION OF THE INVENTION

The present invention provides a method of modulating the release of drug from the outer surface of coatings on substrates by modulating the drying rate of the coatings. Specifically, a method of the present invention comprises providing substrates and preparing mixtures to apply to the substrates. Each of the mixtures comprises a polymer, a solvent, and drug, and the mixtures are applied to respective ones of the substrates to form coatings on the substrates. Each of the coatings has an outer surface. The method further comprises modulating the release rate of the drug from the outer surface of the coatings by drying the solvent of each of the mixtures at different drying rates.

Depending on the initial drug release profile desired, the solvent of each of the mixtures can be dried at different drying rates by increasing or decreasing the drying rates of the solvents of each of the mixtures with respect to one another. For example, if it is desired to increase the release rate of the drug from the outer surface of a coating, the drying rate can be decreased. If it is desired to decrease the release rate of the drug from the outer surface of a coating, the drying rate can be increased. Although not wishing to be bound by theory, it is believed that modulating the drying rate of the solvent affects the nucleation rate of the drug particles, which in turn, affects the size (both diameter and mass) and number of the drug particles, which in turn, affects the release rate of the drug. It is thought that a decrease in the drying rate of the solvent decreases the nucleation rate of the drug particles which increases the size of the drug particles and decreases the number of drug particles on the coating's outer surface. The increase in particle size (even with fewer particles) results in a net increase in mass of drug on the surface of the coating, which increases the release rate of the drug from the coating's outer surface. In contrast, an increase in the drying rate of the solvent is thought to increase the nucleation rate of the drug particles, which decreases the size of the drug particles and increases the number of drug particles on the coating's outer surface, which decreases the release rate of the drug from the coating's outer surface.

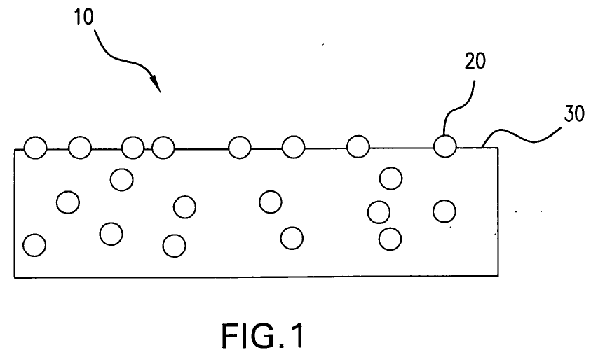

Preferably, in a method of the present invention, the drug and the polymer are both soluble in the solvent but the drug is insoluble in the polymer. Accordingly, once the solubility limit of the drug is reached (during the drying stage of the coating), the drug particles precipitate out from the polymer resulting in spheres of drug particles dispersed throughout the bulk and surface of the polymer coating. Referring to FIG. 1, such phase separation of the drug particles 20 results in discrete domains of drug particles 20 on the outer surface 30 of coating 40 of substrate 10.

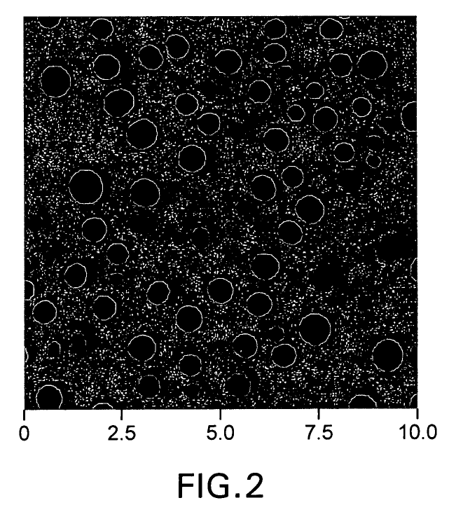

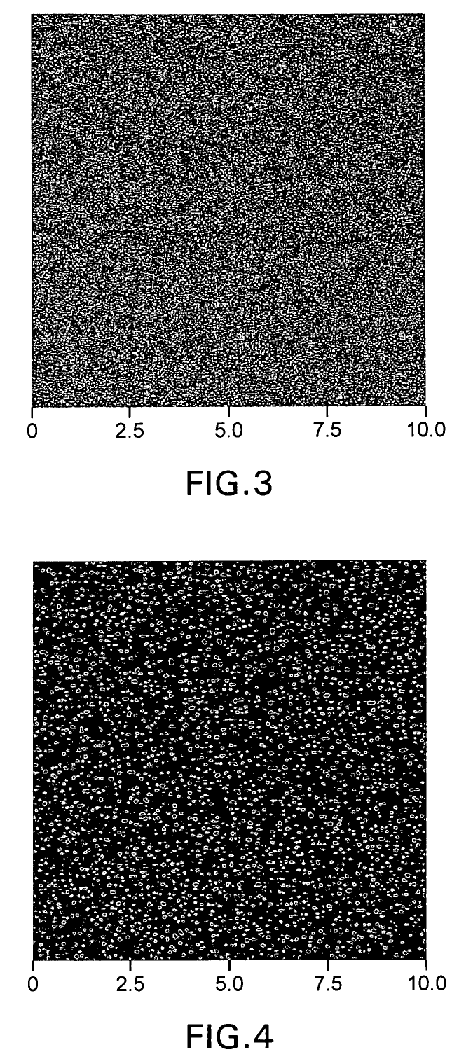

As described further in Example 1 and as illustrated in FIGS. 2-4, drying the solvent of polymer/drug/solvent mixtures at different rates affects the drug particle size (mass) at the outer surface of the coated substrates. Specifically, referring to FIG. 2, a “slow” drying condition (70/30 toluene/THF and no exposure of the coated substrate to forced air) results in average drug diameter of 500 nanometers (nm) on the outer surface of the coated substrate. Referring to FIG. 3, a “fast” drying condition (100% THF and exposure of the coated substrate to forced air) results in average drug diameter of 45 nm on the outer surface of the coated substrate. Such results indicate that lowering the drying rate of the solvent in the mixture applied to a substrate to form a coating on the substrate increases the size of the drug particles on the outer surface of the coating.

As described further in the Example, drying the solvent of polymer/drug/solvent mixtures at different rates affects the release rate of drug from the outer surface of the coated substrates. Specifically, preparing coatings by a solution film cast process at room temperature and at low air flow where the drying rate of the solvent is approximately 5 to 15 seconds, results in a drug particle morphology different than that seen with conventional spray processes, where the drying rate of the solvent is approximately 1/100^{th} to 1/1000^{th} of a second. As illustrated in TABLE 3 of the Example, the average drug particle size generated from a solution film cast process is about 45-500 nm, and the drug particle size generated from a conventional spray process is about 20-50 nm. Initial drug release (over the first 24 hours of release) from solution film cast coatings is about 2-11% and initial drug release from spray coatings is approximately 2.7%, indicating an increase of initial drug release up to about 800% for drug particles released from solution film cast coatings.

Modulating the drying rate of the solvent can be performed by various methods, such as, for example, solvent selection, exposure to air flow, temperature adjustment, adjustment of the percent solid of the mixture, and variation of the coating thickness. For example, if it is desired to increase the drying rate of the solvent, a “fast” drying solvent with an evaporation rate of about 8 or greater compared to n-butyl acetate, which has an evaporation rate of 1, can be used such as, for example THF, diethylether and acetone. Alternatively, to increase the drying rate of the solvent, the mixture applied to the substrate can be exposed to air flow, the chamber temperature can be increased, the percent solids of the mixture can be increased, and/or the coating thickness can be decreased. If it is desired to decrease the drying rate of the solvent, a “slow” drying solvent with an evaporation rate of about 2 or less compared to n-butyl acetate, which has an evaporation rate of 1, can be used such as, for example, xylene, dioxane, and toluene. Alternatively, to decrease the drying rate of the solvent, the chamber temperature can be reduced, the percent solids of the mixture can be decreased, and/or the coating thickness can be increased. The coating can be applied to the substrate by any known method in the art including solution film casting (such as dipping or knife coating), spraying, rolling, brushing, electrostatic plating or spinning, vapor deposition, air spraying including atomized spray coating, and spray coating using an ultrasonic nozzle, so long as the parameters of these processes can be adjusted to modulate the drying rate as desired.

Once a desired drug release rate is obtained from a coating on one of the substrates, medical devices may be manufactured that have coatings that release drug at this desired release rate. Specifically, during the manufacturing process, the solvent of the mixtures applied to the medical devices to form coatings on the medical devices can be dried at the drying rate that corresponds to the desired drug release rate obtained from the respective coated substrate.

The drug in the mixtures applied to the substrates according to the present invention may be any pharmaceutically acceptable therapeutic agents such as non-genetic therapeutic agents, biomolecules, small molecules, or cells.

Exemplary non-genetic therapeutic agents include anti-thrombogenic agents such as heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, cladribine, 5-fluorouracil, methotrexate, doxorubicin, daunorubicin, cyclosporine, cisplatin, vinblastine, vincristine, epothilones, endostatin, trapidil, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneous vascoactive mechanisms; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the drug may be combined to the extent such combination is biologically compatible.

With respect to the type of polymers that may be included in the mixture according to the present invention, such polymers may be biodegradable or non-biodegradable. Non-limiting examples of suitable non-biodegradable polymers include polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; styrene-isobutylene-styrene block copolymers such as styrene-isobutylene-styrene tert-block copolymers (SIBS); polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

In a preferred embodiment, the polymer is a triblock copolymer of PS (end caps) and polyisobutylene.

With respect to other types of solvents that may be used in the mixture according to the present invention, non-limiting examples of suitable solvents include dimethylsulfoxide (DMSO), chloroform, acetone, water (buffered saline), xylene, methanol, ethanol, 1-propanol, tetrahydrofuran, 1-butanone, dimethylformamide, dimethylacetamide, cyclohexanone, ethyl acetate, methylene chloride, methylethylketone, propylene glycol monomethylether, isopropanol, isopropanol admixed with water, N-methyl pyrrolidinone, toluene, and combinations thereof.

Furthermore, multiple types of drug particles, polymers, and/or solvents may be utilized in the mixture according to the present invention.

Non-limiting examples of substrates include polymeric films or medical devices such as catheters, guide wires, balloons, filters (e.g., vena cava filters), stents, stent grafts, vascular grafts, intraluminal paving systems, implants and other devices used in connection with drug-loaded polymer coatings.

### EXAMPLE

### Modulating the Driving Rate of a Solvent in a Coating Comprising a Mixture of a Polymer, a Solvent, and Drug to Affect Drug Particle Size at the Outer Surface of the Coating

A series of six drying conditions are examined to determine the effect on drug particle size and drug release at the outer surface of a coated substrate. For all drying conditions, 25% solid solutions are prepared in mixtures of toluene and THF. Of the solids, 91.2% is polymer and 8.8% is paclitaxel. TABLE 1 shows the solution conditions.

TABLE 1

| Toluene/THF | polymer (g) | paclitaxel (g) | THF (g) | Toluene (g) |
|---|---|---|---|---|
| 0/100 | 5.7 | 0.55 | 18.75 | 0 |
| 50/50 | 5.7 | 0.55 | 9.37 | 9.37 |
| 70/30 | 5.7 | 0.55 | 5.62 | 13.12 |

TABLE 2 shows the parameters of the six drying conditions and ranks the conditions from fastest drying condition to slowest drying condition. The drying rate is adjusted by varying the ratio of toluene (“slow” evaporating solvent) and THF (“fast” evaporating solvent) and by introducing forced air across the surface of the coating during the drying stage.

TABLE 2

| Relative Drying Rate | Toluene/THF | Air Flow | Condition # |
|---|---|---|---|
| FASTEST | 70/30 | NO | 1 |
| | 50/50 | NO | 2 |
| | 70/30 | YES | 3 |
| | 50/50 | YES | 4 |
| | 0/100 | NO | 5 |
| SLOWEST | 1/100 | YES | 6 |

For all drying conditions, the polymer, paclitaxel, and solvent are added into a glass bottle and mixed on a rotational mixer to allow the polymer and paclitaxel particles to dissolve. Each resultant mixture is coated on a 0.005 inch thick polyethylene terephthalate (PET) film using a knife coater (BYK Gardner) at a wet gap setting of 5.5 mm. For conditions in which forced air is used room temperature forced air is applied across the surface of the coated substrates during the drying stage. Air flow is at 80 standard cubic feet per minute. For conditions using no forced air the coating is allowed to dry in ambient air at room temperature. After all the coatings are “touch dry,” they are dried further at 65° C. for 30 minutes and then for 3 hours at 70° C. under vacuum to remove residual solvent. Coating thickness under all drying conditions is 20 μm. Atomic force microscopy images are taken of all coated substrates under all drying conditions.

FIG. 2 is the atomic force microscopy (AFM) image of the coated substrate under the slowest drying condition (condition 1), and average paclitaxel diameter under such a drying condition is 500 nm. FIG. 3 is the AFM image of the coated substrate under the fastest drying condition (condition 6), and average paclitaxel diameter under such a drying condition is 45 nm. FIG. 4 is the AFM image of the coated substrate under an intermediate drying condition (condition #4), and average paclitaxel diameter under such a drying condition is 76 nm. Results indicate that decreasing the drying rate of the solvent increases the drug particles size at the outer surface of the coated substrate.

A kinetic drug release test (KDR) is performed by incubating the coated samples in a media containing IPA/water/surfactant. The media is removed at various time points and analyzed by high performance liquid chromatography to determine the quantity of drug eluted from the coating. TABLE 3 depicts particle diameter of the paclitaxel particles and KDR results at different drying conditions and indicates that decreasing the drying rate of the solvent increases the diameter of the paclitaxel particles, which increases the cumulative release of the paclitaxel particles from the surface of coated substrates.

TABLE 3

| | | Average # | | |
|---|---|---|---|---|
| | | Drug | Percent | Percent |
| | | Particles | Paclitaxel | Paclitaxel |
| | Average | per 100 sq | Release | Release |
| | Paclitaxel | microns of | Over 4 | Over 24 |
| | Diameter | surface | Hour | Hour |
| Drying Conditions | (nanometers) | coating | Period | Period |
| 70/30 toluene/THF | 500 | 99 | 10.0 | 10.7 |
| 70/30 toluene/THF | 212 | 162 | 3.8 | 4.4 |
| with exposure to air | | | | |
| flow | | | | |
| 50/50 toluene/THF | 304 | 974 | 4.0 | 5.0 |
| 50/50 toluene/THF | 76 | 2121 | 0.96 | 1.8 |
| with exposure to air | | | | |
| flow | | | | |
| 0/100 toluene/THF | 73 | 1493 | 0.69 | 1.4 |
| 0/100 toluene/THF | 45 | 1777 | 0.57 | 1.3 |
| with exposure to air | | | | |
| flow | | | | |
| Standard spray | 20-50 | | 0.74 | 2.3 |
| process (DES) | | | | |

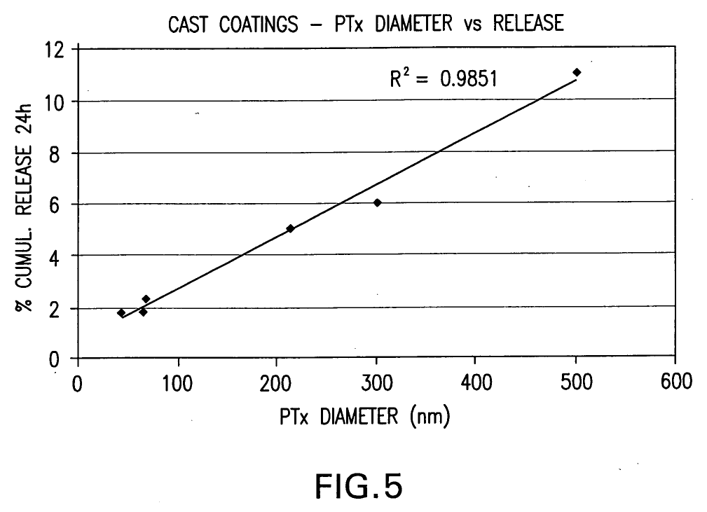

Initial drug release (over the first 4 hours of release) from solution film cast coatings ranges from approximately 0.7-10% and initial drug release from spray coatings (same coating composition) is approximately 2.3%, indicating an increase of initial drug release of about 1200% for paclitaxel particles released from slow dried solution film cast coatings compared to spray coatings. FIG. 5 depicts a chart of paclitaxel particle diameter on the surface of the coated substrates versus cumulative release of the paclitaxel drug particles over a 24 hour period. FIG. 5 indicates that as the paclitaxel drug particle diameter increases, the percent cumulative release increases.

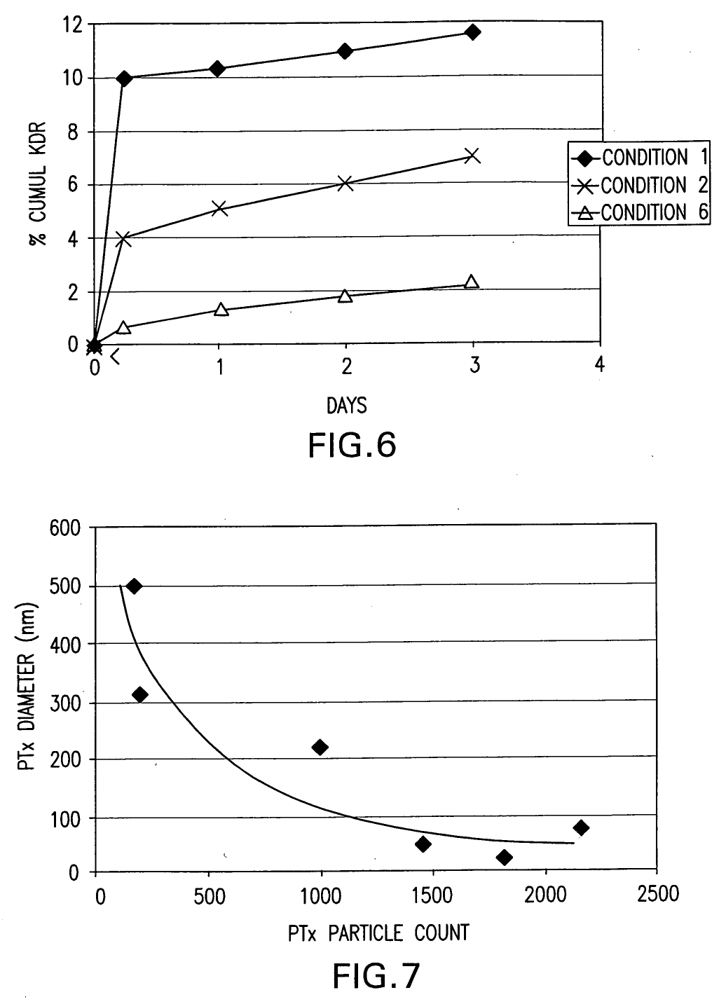

FIG. 6 shows the percent cumulative drug release over a three day period. The diameter of the drug particles at the surface of the coating impacts the initial “burst” release (release at 0-4hr ). This burst is due to dissolution of the drug exposed at the outer surface of the coating. Dissolution of drug from the bulk of the coating is slower due to the fact that it is encased in the polymer matrix. The rate of release from the bulk of the coating is independent of the particle diameter—this corresponds to time points after about 1 day (the slope of the release curves from 4hr—3days are similar for the three conditions.

FIG. 7 depicts a plot of paclitaxel particle diameter vs particle count (# drug particles/unit surface area). In general, the number of particles on the surface decrease with decreasing drying rate. Thus as the drying rate is reduced there are fewer but larger drug particles at the surface.

The foregoing description and examples have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

I claim:

1. A method of modulating drug release from coatings on substrates comprising:
providing substrates;
preparing mixtures, each of the mixtures comprising a polymer, a solvent, and drug;
applying each of the mixtures to respective ones of the substrates to form coatings on the substrates, each of the coatings having an outer surface; and
modulating the release rate of the drug from the outer surface of the coatings by drying the solvent of each of the mixtures at different drying rates.

2. The method of claim 1, wherein the drug comprises drug particles that are insoluble in the polymer.

3. The method of claim 1, wherein applying each of the mixtures to respective ones of the substrates comprises applying at least one of the mixtures to a respective one of the substrates by solution film casting.

4. The method of claim 1 wherein drying the solvent of each of the mixtures at different drying rates comprises decreasing the drying rate of the solvent of at least one of the mixtures to increase the release rate of the drug from the outer surface of at least one of the coatings.

5. The method of claim 4, wherein the drying rate of the solvent is decreased by using a solvent that has an evaporation rate of about 2 or less compared to the evaporation rate of n-butyl acetate.

6. The method of claim 5, wherein the solvent is toluene.

7. The method of claim 4, wherein decreasing the drying rate of the solvent decreases a nucleation rate of particles of the drug.

8. The method of claim 4, wherein decreasing the drying rate of the solvent increases a diameter of particles of the drug.

9. The method of claim 4, wherein decreasing the drying rate of the solvent results in an a decrease in the number of particles of the drug on the outer surface of the at least one of the coatings.

10. The method of claim 1, wherein drying the solvent of each of the mixtures at different drying rates comprises increasing the drying rate of the solvent of at least one of the mixtures to decrease the release rate of the drug from the outer surface of at least one of the coatings.

11. The method of claim 10, wherein the drying rate of the solvent is increased by passing an air stream over the mixture applied to the at least one of the coatings.

12. The method of claim 10, wherein the drying rate of the solvent is increased by using a solvent with an evaporation rate of about 8 or greater compared to the evaporation rate of n-butyl acetate.

13. The method of claim 12, wherein the solvent is THF.

14. The method of claim 10, wherein increasing the drying rate of the solvent increases the nucleation rate of particles of the drug.

15. The method of claim 10, wherein increasing the drying rate of the solvent decreases a diameter of particles of the drug.

16. The method of claim 10, wherein increasing the drying rate of the solvent results in an increase in the number of the particles of drug on the outer surface of the at least one of the coatings.

17. The method of claim 1, wherein the substrate is a polymeric film.

18. The method of claim 1, wherein the substrate is a medical device.

19. The method of claim 18, wherein the medical device is a stent.
